(19) United States
(12) Patent Application Publication
(10) Pub. No.: US 2004/0224822 A1
(43) Pub. Date: Nov. 11, 2004

(54) Timer for breathing exercises

(76) Inventor: Johann Brandt Verheem, Carlsbad, CA (US)

Correspondence Address: Johann Brandt Verheem, 3021 Camino Serbal, Carlsbad, CA, 92009, US

(21) Appl. No.: 10/839,998

(22) Filed: May 5, 2004

Related U.S. Application Data
(60) Provisional application No. 60/468,064, filed on May 6, 2003.

(51) Int. Cl. A62B 7/04; A63B 71/00; A61M 16/00

(52) U.S. Cl. 482/8; 482/1

(57) ABSTRACT

Disclosed is an electronic timer device that provides the user with audible, visible or sensory signals indicating when to inhale, when to exhale and when to hold breath during breathing exercises. The length of each inhalation, exhalation and holding of breath can be modified and the ratio between inhaling, exhaling and holding breath can be modified to provide breathing exercises with various different physiological benefits.

This application is a utility patent application that claims the benefit of U.S. Provisional Application Serial No. 60/468,064 filed on May 06, 2003, which is hereby fully incorporated herein as though set forth in full.

### FIELD OF THE DISCLOSURE

The present invention relates to a timing device used for pacing of human breathing for the purpose of deriving health benefits from monitoring and applying specific time intervals to the inhalation, exhalation and holding of breath. By engaging in breathing at specific variable intervals and ratios, the user can obtain health benefits, such as relaxation, avoiding insomnia, aerobic exercise and reduction in cravings for cigarettes.

### BACKGROUND OF THE DISCLOSURE

Breathing exercises have been a part of many different forms of exercise and therapy for maintaining and treating health and wellness. Ancient techniques of meditation and yoga, as well as modern forms of exercise, meditation and forced breathing are widely accepted and scientifically, well-documented techniques with health benefits. These techniques all rely on altering the normal breathing pattern, the length of the inhale and exhale cycle and the ratios between inhaling, holding the breath and exhaling. While the benefits of altered breathing are well accepted, the practice of altered breathing is difficult for the user of these techniques. As a result of the fact that breathing is mostly an involuntary activity, the user of altered breathing techniques has to focus on instructions provided by an instructor, a video or has to count his or her own breathing cycles.

### SUMMARY OF THE DISCLOSURE

Disclosed is an electronic timer device that transmits signals to a user through audible, visible or sensory stimuli generated through applying pressure through pressure points on the skin or through a small electric current touching the skin, for the purpose of assisting the user in pacing and altering the cycles of inhaling, exhaling and holding breath. The stimuli or recognizable signals inform the user when to inhale and for how long a period of time to inhale, when to hold the breath after inhalation and for how long, when to exhale and for how long, as well as when to hold after exhaling and for how long. The device further allows the user to alter the ratios between the inhalation, exhalation and holding of breath cycles and furthermore allows the user to adjust the length of each of the inhalation, exhalation and holding breath cycles. Various cycles of inhaling, exhaling and holding breath provide different physiological benefits, such as relaxation, boosting energy levels, increasing caloric metabolism, increasing mental acuity and assisting the user with falling asleep. The device can be worn on the body of the user, around the wrist, the ankle or around the abdomen.

### BRIEF DESCRIPTION OF THE DRAWINGS

The invention will now be described in greater detail with reference to the preferred embodiments illustrated in the accompanying drawings, in which like elements bear like reference numerals and wherein:

FIG. 1 is a front perspective showing the placement of visible lights and controls on the device.

FIG. 2 is a front perspective of the timing device worn around the wrist of a user.

FIG. 3 is a side perspective of the device showing the protruding pressure pins.

FIG. 4(a), 4(b) and 4(c) are side perspectives of the device showing three possible combinations of protruding and detracting of the pressure pins.

FIG. 5 is a front perspective showing the device worn around the ankle of a user.

FIG. 6 is a front perspective showing the device worn around the abdomen of a user.

### DETAILED DESCRIPTION OF THE DISCLOSURE

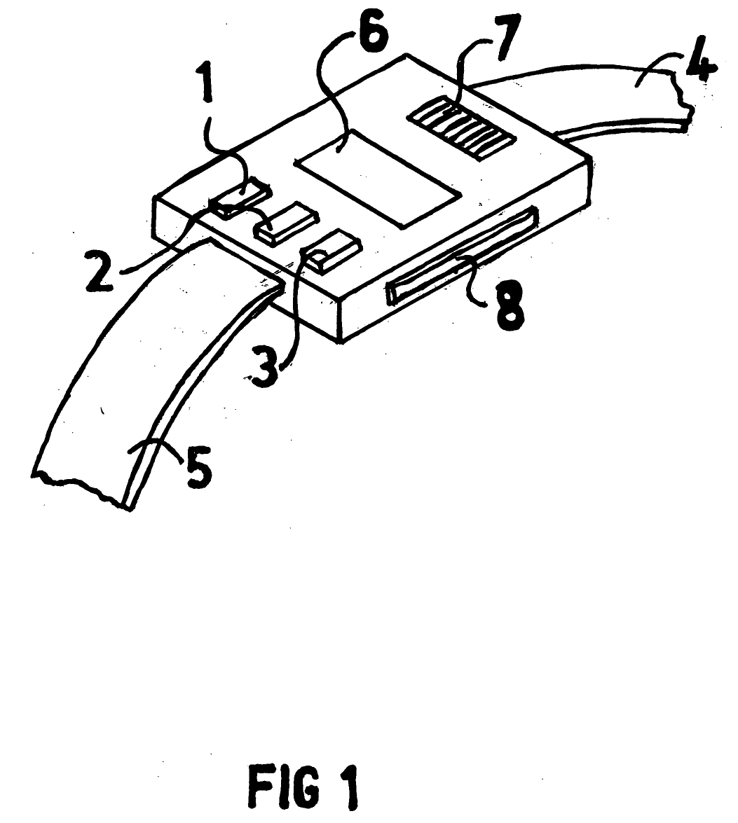

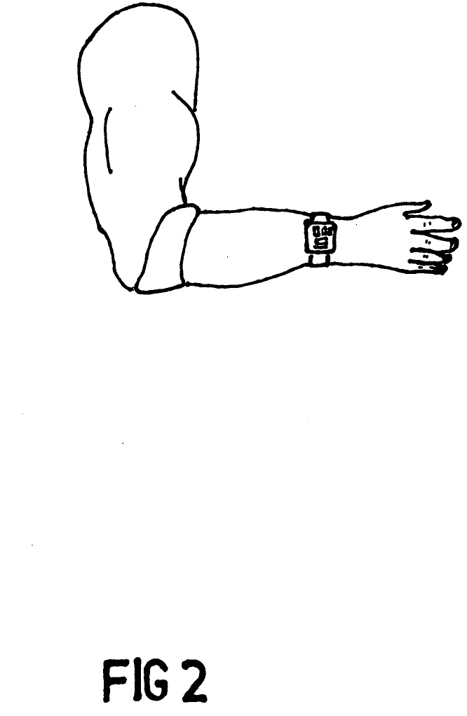

Disclosed is an electronic timing device that is compact, lightweight and can be worn on a user's wrist in similar fashion as a wrist watch on the inside or outside of the wrist, or around the ankle or mid section of the abdomen. The device as shown in FIG. 1 through FIG. 6 shows a device that incorporates an electronic timing device that provides a signal for the user to determine when to inhale and for how long to inhale, when to exhale and for how long to exhale, and when to hold breath between inhale and exhale and for how long to hold breath.

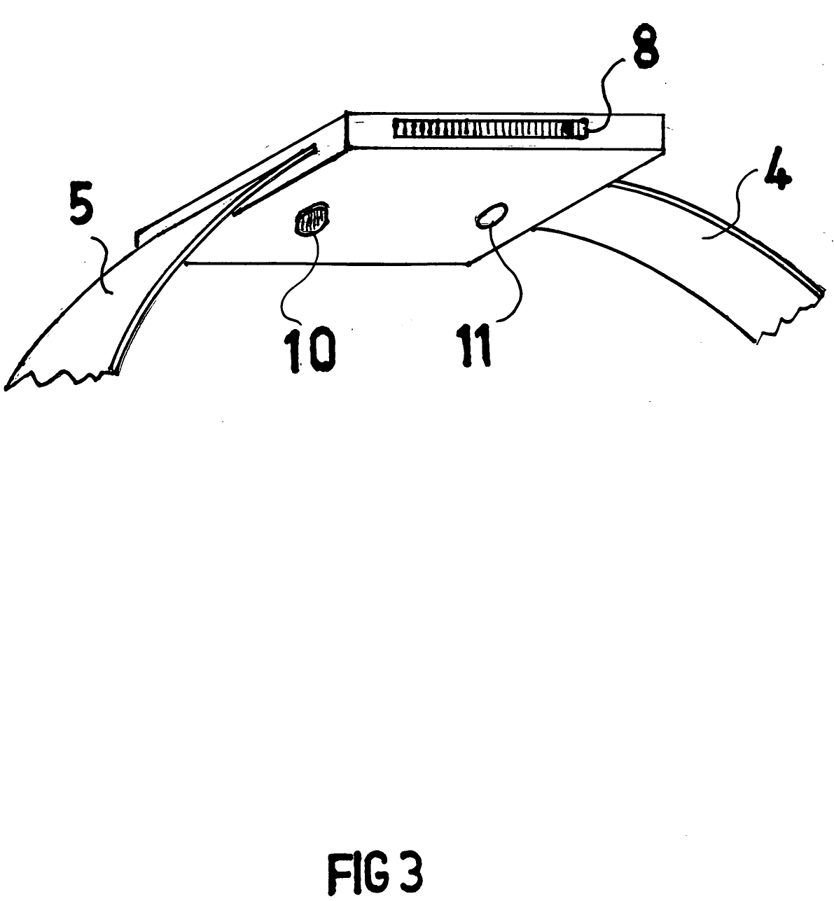

The device in FIG. 1 shows an LCD screen (6) that demonstrates the specific breathing exercise that will run as soon as the user presses the control button (8), selects a breathing program and sets a difficulty level. As soon as the user starts the program, the device will provide a signal on when to inhale, when to exhale and when to hold breath in by providing a distinct sound for each of the actions, by one of the indicator lights (1, 2 or 3) coming on and staying on for the duration of the inhalation, exhalation or holding of breath, or, as shown in FIG. 3, by applying pressure to the user's skin through one of the protruding and detracting pressure points (10 and 11) that protrudes or retracts during the exercise.

Any one of the three signals (light, sound or pressure) can be used independently or any of the three can be used in combination. Breathing exercises are done in variable ratios between the inhalation, holding and exhalation of breath. As an example, a typical breathing exercise would let the participant inhale for 4 seconds, hold breath for 2 seconds, exhale for 6 seconds and hold for 2 seconds. The device allows the user to select from various ratios between the actions of inhaling, exhaling and holding of breath. The ratio in the example above would be 4:2:6:2. The ratio can be changed to many different ratio combinations to assist with relaxing, sleeping, energizing, focusing or exercising. Ratios can be of various combinations and lengths. For instance, the example above of 4:2:6:2, where 4 indicates 4 seconds of inhaling, 2 indicates 2 seconds of holding, 6 indicates 6 seconds of exhaling, 2 indicates 2 seconds of holding before the cycle repeats, can be changed, for example, to 6:2:4:2, or can be increased or decreased to, for example, 6:3:9:3. An increase in the length of each of the four cycles would be considered an increase in difficulty level.

The various different ratios for different purposes and the length of the ratios can be adjusted by the control button (8) by selecting the specific exercise as displayed on the LCD screen (6) and by selecting the difficulty level as displayed by the LCD screen (6).

Exercises are started by setting the specific exercise and difficulty level and by pressing the control button (8) to begin the exercise. The exercise will continue for a period of time and automatically stop, such as would be useful for the treatment of insomnia, or could be manually stopped by pressing the control button (8). While a selected exercise is running, the indicator lights (1, 2 and 3) will flash for the full period of inhalation, exhalation and holding. Each of the indicator lights can be a different color, or one light with 3 different colors can be used. The LCD screen (8) provides a training mode that shows the user, in text, which action to take while a specific light is on. As an example, while the device is in training mode, as selected by the control button (8), light (1) is a different color and indicates inhalation. For the full time that light (1) is on, the LCD screen (6) will display “inhale”. Similarly, for the full time that light (2) is on, the LCD screen will display “hold” and for the full time light (3) is on, the LCD screen will display the word “exhale”.

In a similar fashion to the lights indicating the various ratios and times of inhalation, exhalation and holding of breath, unique electronic sounds can be transmitted through the speaker (7) at different tones, indicating to the user when and for how long to inhale, when and for how long to exhale and when and for how long to hold breath after inhaling or exhaling.

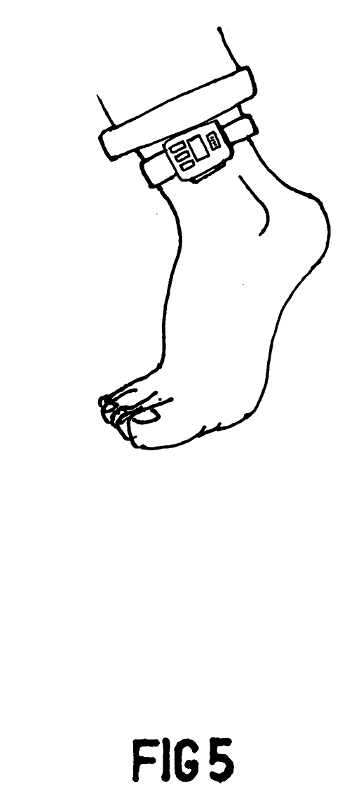

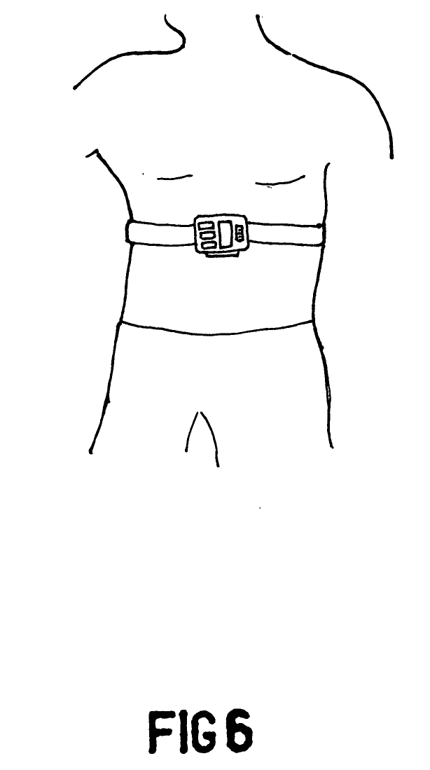

The bottom of the body of the device is placed against the user's skin, either by strapping the device around the wrist of the user as shown in FIG. 1, or by strapping the device using a different strap around the ankle of the user as shown in FIG. 5, or by strapping the device using a longer strap around the abdomen as shown in FIG. 6.

The pressure applicators (10 and 11) protrude and detract from the bottom of the device where it touches the user's skin. The pressure applicators are mechanically driven through electrical means or electromagnetically activated means to protrude or detract, thereby providing the user with a signal, based on three possible positions of the pressure applicators.

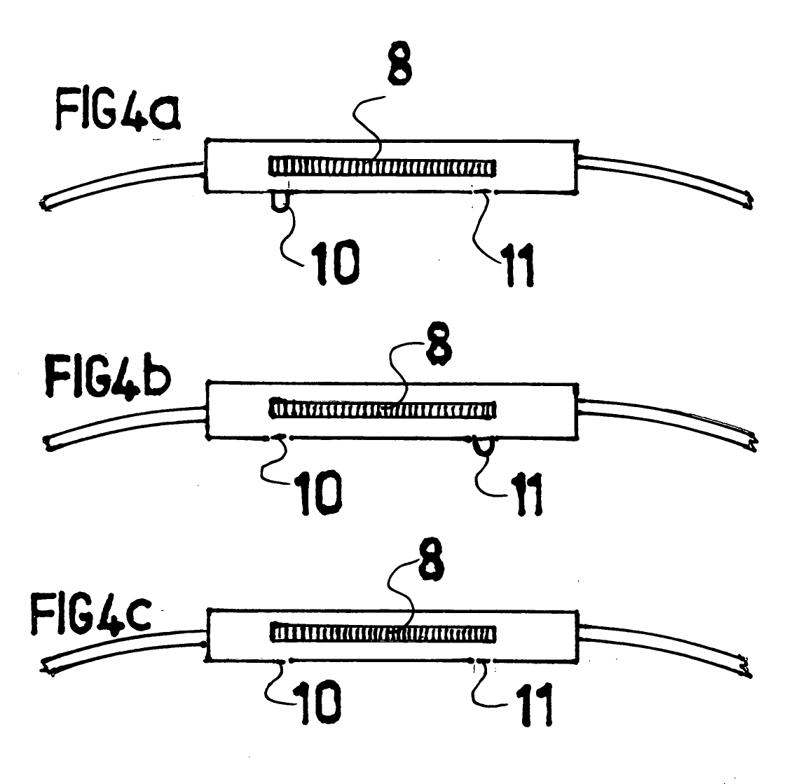

During inhalation, pressure applicator (10) will be in protruded position as shown in FIG. 4(a). This will indicate to the user that for the full time the pressure applicator is protruded, the user should inhale. During exhalation, the pressure applicator (11) will be protruded and shown in FIG. 4(b), for the full period of the exhalation cycle. During the processing of holding breath, both pressure applicators (10 and 11) will be detracted, as shown in FIG. 4(c). By providing a sensory touch indicator of whether to inhale, exhale or hold, the user can do breathing exercises at the suggested ratios and difficulty levels without having to look at the lights (1, 2 and 3) or the LCD display (6), or to listen to the sounds produced by the speaker (7).

While the preferred embodiment of the present invention has been shown and described, it will be apparent to those skilled in the art that various modifications may be made in the embodiment without departing from the spirit of the present invention. Such modifications are all within the scope of the present invention.

1. An electronic timer device, compromising:
A device with electronic components allowing the user to receive an audible, visible or sensory signal, or any combination of these signals, indicating when to inhale, when to exhale and when to hold breath.

2. The electronic timer device of claim 1 where the device is adapted to enable the user to wear the device on his or her body.

3. The electronic timer device of claim 1 where the device is adapted so that the user can wear the device around his or her wrist.

4. The electronic timer device of claim 1 where the device is adapted so that the user can wear the device around his or her midsection.

5. The electronic timer device of claim 1 where the device is adapted so that the user can wear the device around one of his or her ankles.

6. The electronic timer device of claim 1 where the device is adapted to provide different perceivable signals to the user for inhaling, exhaling and holding breath during breathing exercises.

7. The electronic timer device of claim 1 where the device is adapted to provide the perceivable signals to the user for inhaling, exhaling and holding breath during breathing exercises by means of one or more lights.

8. The electronic timer device of claim 1 where the device is adapted to provide the perceivable signals to the user for inhaling, exhaling and holding breath during breathing exercises by means of two or more different colored lights (1, 2 and 3)

9. The electronic timer device of claim 1 where the device is adapted to provide the perceivable signals to the user for inhaling, exhaling and holding breath during breathing exercises by means of two or more different sounds transmitted through a speaker.

10. The electronic timer device of claim 1 where the device is adapted to provide the perceivable signals to the user for inhaling, exhaling and holding breath during breathing exercises by means of three different visual indicators displayed on a LCD screen.

11. The electronic timer device of claim 2 where the device is adapted to provide the perceivable signals to the user for inhaling, exhaling and holding breath during breathing exercises by means of one or more mechanically activated pressure points (10 and 11) protruding and retracting from the device where it touches the human skin, thereby providing the user with sensory feedback on when to inhale and for how long to inhale, when to exhale and for how long to exhale and when to hold breath and for how long to hold breath.

12. The electronic timer device of claim 2 where the device is adapted to provide the perceivable signals to the user for inhaling, exhaling and holding breath during breathing exercises by means of one or more low voltage electrical currents touching the user's skin, thereby providing the user with sensory feedback on when to inhale and for how long to inhale, when to exhale and for how long to exhale and when to hold breath and for how long to hold breath.

13. The electronic timer device of claim 1 where the user can adjust the length of time of each signal indicating inhaling, exhaling and holding breath.

14. The electronic timer device of claim 8 where the user can adjust the length of time that each signal, provided by at least one light, lasts.

15. The electronic timer device of claim 8 where the user can adjust the length of time that each signal, provided by two or more lights, lasts.

16. The electronic timer device of claim 9 where the user can adjust the length of time that each signal, provided by sound generated by the speaker (7), lasts.

17. The electronic timer device of claim 10 where the user can adjust the length of time that each visual instruction, provided by LCD display (6), lasts.

18. The electronic timer device of claim 11 where the user can adjust the length of time that each pressure signal, provided by mechanical pressure points (10 and 11), lasts.

19. The electronic timer device of claim 12 where the user can adjust the length of time that each signal, provided by the low voltage electrical current, lasts.

20. The electronic timer device of claim 1 where the user can adjust the ratio between the length in time of the signals providing instructions to inhale, exhale and hold breath.

21. The electronic timer device of claim 7 where the user can adjust the ratio between the length in time of the signals provided by at least one light providing instructions to inhale, exhale and hold breath.

22. The electronic timer device of claim 8 where the user can adjust the ratio between the length in time of the signals provided by 2 or more lights indicating when to inhale, exhale and hold breath.

23. The electronic timer device of claim 9 where the user can adjust the ratio between the length in time of the signals provided by 2 or more differentiated sounds from the speaker (7) indicating when to inhale, exhale and hold breath.

24. The electronic timer device of claim 10 where the user can adjust the ratio between the length in time of the signals provided by LCD display (6) indicating when to inhale, exhale and hold breath.

25. The electronic timer device of claim 11 where the user can adjust the ratio between the length in time of the signals provided by 2 or more pressure points making contact with the user's skin, indicating when to inhale, exhale and hold breath.

26. The electronic timer device of claim 12 where the user can adjust the ratio between the length in time of the signals provided one or more low voltage electrical currents indicating when to inhale, exhale and hold breath.

27. The electronic timer device of claim 1 where the user can adjust the ratio between the length in time of the signals providing instructions to inhale, exhale and hold breath.

28. The electronic timer device of claim 1 where the user can adjust the ratio between the length in time of the signals providing instructions to inhale, exhale and hold breath.

29. The electronic timer device of claim 1 that has an on and off power switch to initiate or terminate signals for breathing exercises.

30. The electronic timer device of claim 1 that provides for automatic turn off after an amount of time has expired from turning on the device.

31. The electronic timer device of claim 1 that incorporates the functions of time of day and date typically found in wrist watches.
